(19) United States
(12) Patent Application Publication
(10) Pub. No.: US 2002/0164574 A1
(43) Pub. Date: Nov. 7, 2002

(54) Methods for utilization of microplates in chemical classification and analysis

(76) Inventors: Matthew M. Tanzer, Durham, NC (US); John Hamer, Durham, NC (US)

Correspondence Address: PARADIGM GENETICS, INC, 108 ALEXANDER DRIVE, P O BOX 14528, RTP, NC, 27709-4528, US

(21) Appl. No.: 09/850,608

(22) Filed: May 7, 2001

(51) Int. Cl. C12Q 1/00; C12Q 1/68; G01N 33/53; C12Q 1/18

(52) U.S. Cl. 435/4; 435/7.1; 435/32

(57) ABSTRACT

The growth of a cell is measured in a microplate format in the absence or presence of a test compound, across a variety of different environmental conditions, such as the presence of carbon sources, different pH, nitrogen sources, salinity, etc. The patterns of growth across a number of different parameters is assembled into a dataset, which dataset may then be utilized for information about the pathway or pathways that are affected by the compound. Unknown compounds may be identified by matching the characteristic growth patterns to the growth patterns from known compounds, or to classes of compounds. Alternatively, unknown or known compounds, including biologically active compounds and derivatives and analogs of biologically active compounds, are classified according to the particular pattern of growth.

### FIELD OF THE INVENTION

The invention relates to methods for screening for modulators of cell processes, which provide multiparameter information about the action of the compounds tested.

### BACKGROUND OF THE INVENTION

Despite recent advances in methods for identification, cloning, and characterization of genes and genomes, much remains to be learned about cell physiology in general, including how cells produce natural products; mechanisms for resistance to cytotoxic or inhibitory agents; elucidation of genes involved in specific biosynthetic pathways; and genes involved in environmental tolerance, e.g., salt tolerance, drought tolerance, or tolerance to anaerobic conditions.

In addition to basic information about cell physiology, the effectiveness and mode of action of anti-microbial drugs remains an important question. Even before the development of the first antibiotics, bacteria had demonstrated an ability to adapt to stress in the environment, resulting in the development of resistance. In recent years, the variety of antimicrobial agents has increased substantially, along with a parallel increase in resistant pathogenic microorganisms. Resistance is now recognized against all clinically available antimicrobial agents. The response to antimicrobial resistance in the medical community has been to use new or alternative antibiotics not previously used against the resistant bacteria. This approach has required the continuous development of new antibiotics, either as modifications of currently existing compounds or as combinations of compounds that may inhibit or bypass the bacterial resistance mechanisms.

The evaluation of fungal-specific agents is also of interest. Not only are fungal-specific therapeutics difficult to identify, but many of the drugs currently available for treatment of mycoses have significant side effects or lack effectiveness against some important pathogens. Also, some evidence exists for the development of resistance to these drugs. There is therefore an ongoing need for novel antifungal drugs with few side effects and with effectiveness against pathogens for which current drugs are inadequate.

Fungal pathogens often are naturally resistant to many therapeutics by virtue of cellular permeability barriers to drug entry. Development of fungicide resistance occurs when a fungal cell or a fungal population that originally was sensitive to a fungicide becomes less sensitive by heritable changes after a period of exposure to the fungicide. Most instances of resistance are related to a change at the site of action or a change in the uptake of the fungicide, with detoxification being a rare event. In certain applications, e.g. agriculture, it is possible to combat resistance through alternation of fungicides or the use of fungicide mixtures. To prevent or delay the buildup of a resistant pathogen population, different chemicals that are effective against a particular disease must be available. One way of increasing the number of available chemicals is to search for new site specific inhibitors. Thus, the challenge is to develop methods for identifying compounds which can penetrate the pathogen and specifically kill it or arrest its growth without also adversely affecting the plant host.

Classical approaches for identifying antimicrobial compounds have relied almost exclusively on inhibition of growth as an endpoint. Libraries of natural products, semi-synthetic, or synthetic chemicals are screened for their ability to kill or arrest growth of the target pathogen or a related nonpathogenic model organism. These tests are cumbersome and provide no information about a compound's mechanism of action. The promising lead compounds that emerge from such screens must then be tested for possible toxicity to the human, animal, or plant host, and detailed mechanism-of-action studies must subsequently be conducted to identify the affected molecular target and precisely how the drug interacts with this target.

The rewards for identification of a useful agents can be enormous, but the percentage of hits from any screening problem are generally very low. Desirable compound screening methods solve this problem by both allowing for a high throughput so that many individual compounds can be tested; and by providing biologically relevant information so that there is a good correlation between the information generated by the screening assay and the pharmaceutical effectiveness of the compound. Some of the more important features for effectiveness are specificity for the targeted cell or disease, a lack of toxicity at relevant dosages, and specific activity of the compound against its molecular target. Therefore, one would like to have a method for screening compounds or libraries of compounds that allows large numbers of simultaneous assays, where the assay predicts these aspects of future performance.

### SUMMARY OF THE INVENTION

Methods are provided for the classification, identification, and/or mode of action of chemical compounds in high throughput assays. Compounds that act on microbial cells are of particular interest. A cell's ability to grow in the presence of nutrient sources and other environmental conditions is measured in a microplate format in the absence or presence of a test compound. The ability or inability of the cell to grow in the presence of the test compound under different conditions provides information about the pathway or pathways that are affected by the compound, thereby providing for mode-of-action determination. Unknown compounds may be identified by matching the characteristic growth patterns to a known reference pattern. Alternatively, unknown or known compounds, including derivatives and analogs of biologically active compounds, are classified according to the particular type of growth pattern in the presence of a test compound.

Thus, in one aspect, the invention provides a method for characterization of a test compound, the method comprising:

contacting a cell with said test compound in the presence of a plurality of assay conditions, each comprising culture medium and a substrate for growth of said cell;

recording the growth or substrate utilization of said cell in the presence of said test compound; and

deriving a test pattern from the output signal resulting from contact between said cell and said test compound, wherein said test pattern indicates the phenotype of said cell in the presence of said test compound.

In a preferred embodiment, the test compound is an anti-microbial compound, where the concentration of the compound in the assay wells is based on a pre-determined minimal inhibitory concentration (MIC).

In another aspect, the invention provides a method for characterization of a polynucleotide sequence, the method comprising:

genetically modifying a cell with said polynucleotide sequence;

contacting at least one of said genetically modified cell with a plurality of assay conditions, each comprising culture medium and a substrate for growth of said cell;

recording the growth or substrate utilization of said cell; and

deriving a test pattern from the output signal resulting from contact between said cell and said assay conditions, wherein said test pattern indicates the phenotype of said cell resulting from genetic modification with said polynucleotide sequence.

In still another aspect, the invention provides a database of reference patterns, said database comprising a plurality of reference patterns obtained for one or more test compounds by the method of:

contacting at least one cell with said test compound in the presence of a plurality of assay conditions, each comprising culture medium and a substrate for growth of said cell;

recording the growth or substrate utilization of said cell in the presence of said test compound; and

deriving a test pattern from the output signal resulting from contact between said cell and said test compound, wherein said test pattern indicates the phenotype of said cell in the presence of said test compound.

### BRIEF DESCRIPTION OF THE DRAWINGS

FIG. 1 shows the minimal inhibitory concentration (MIC) determination in microtiter plate assay. [Hi] indicates the inhibitory concentration and [Lo] indicates the sub-inhibitory concentration used for subsequent experiments. [Hi] corresponds to 2× to 10×MIC. [Lo] corresponds to 2 to 4 fold less than the maximum concentration at which no growth inhibition was observed. A subscript of M indicates M. grisea data; a subscript of S indicates S. cerevisiae data. A concentration of 0 contains the fungicide solvent only.

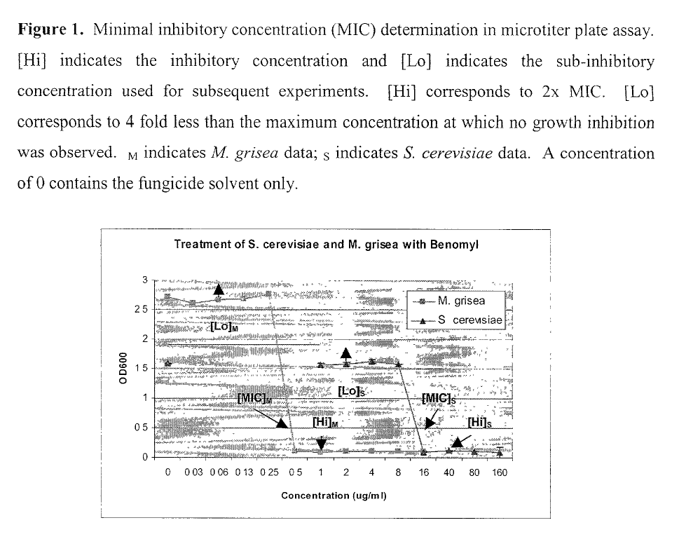

FIG. 2 shows the treatment of S. cerevisiae with fungicides during growth in YT MicroPlate™ (Biolog, Inc.) Data is represented as control vs. fungicide treatment. This initial experiment shows that fenpiclonil shows significant changes in the utilization of glucose, raffinose, and sucrose as compared to the controls. The other two fungicides show no significant differences as compared to controls.

### DETAILED DESCRIPTION OF THE EMBODIMENTS

The methods of the invention are a valuable tool that can aid in the selection and prioritization of compounds from large compound collections for biological testing. The effect of a compound on the growth of cells can be classified in terms of the mechanism of action. The method of the invention has various applications including, but not limited to: drug discovery, identification of the target/mechanism of candidate drug action, facilitation of SAR (structure, action, and reaction) studies, rational approaches to efficacy improvement, and rapid categorization of compounds as to possible therapeutic applications and potential toxicity. The method of the invention can also provide a means to identify genes that play a role in, or that can influence, a selected biological process or pathway.

The growth of a microorganism is measured in a microplate format in the absence or presence of a test compound, across a variety of different environmental conditions, such as the presence of carbon sources, different pH, nitrogen sources, salinity, etc. The patterns of growth across a number of different parameters is assembled into a dataset, which dataset may then be utilized for information about the pathway or pathways that are affected by the compound. Unknown compounds may be identified by matching the characteristic growth patterns to the growth patterns from known compounds, or to classes of compounds. Alternatively, unknown or known compounds, including derivatives and analogs of biologically active compounds, are classified according to the particular pattern of growth.

Thus, the invention provides a method for characterization of a test compound, the method comprising:

contacting at least one cell with said test compound in the presence of a plurality of assay conditions, each comprising culture medium and a substrate for growth of said cell;

recording the growth or substrate utilization of said cell in the presence of said test compound; and

deriving a test pattern from the output signal resulting from contact between said cell and said test compound, wherein said test pattern indicates the phenotype of said cell in the presence of said test compound.

Any compound may be tested in the methods of the invention. Test compounds of particular interest are organic molecules. A preferred embodiment is the use of the methods of the invention to select antimicrobial or anti-fungal compounds. The term “antimicrobial” is used in reference to any compound which inhibits the growth of, or kills microorganisms. It is intended that the term be used in its broadest sense, and includes, but is not limited to compounds such as antibiotics which are produced naturally or synthetically. It is also intended that the term includes compounds and elements that are useful for inhibiting the growth of, or killing microorganisms. In a preferred embodiment, the test compound is an anti-microbial compound, where the concentration of the compound in the assay wells is based on a pre-determined minimal inhibitory concentration (MIC).

Candidate agents generally comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Test compounds include all of the classes of molecules described above, and may further comprise samples of unknown content. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 □l to 1 ml of a biological sample is sufficient.

Compounds and candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Any prokaryotic or eukaryotic cell that can be grown in liquid culture or gelled culture media can be used in the methods of the invention, including, but not limited to bacterial and fungal cells, plant cells, cells from vertebrate and mammalian organisms, and the like. Cells of particular interest are from single or multicellular organisms that are pathogenic for plants or animals.

The range of cell types that can be tested using the methods and compositions of the present invention includes cells that undergo complex forms of differentiation filamentation, sporulation, etc.

Bacterial groups of interest include, but are not limited to, Aquificales, CFB/Green sulfur bacteria group; Chlamydiales/Verrucomicrobia group; Chrysiogenes group; Coprothermobacter group; Cyanobacteria (blue-green algae); Dictyoglomus group; Fibrobacter/Acidobacteria group; Firmicutes (gram-positive bacteria); Flexistipes group; Fusobacteria; Green non-sulfur bacteria; Nitrospira group; Planctomycetales, Proteobacteria (purple non-sulfur bacteria), Spirochaetales; Synergistes group, Thermodesulfobacterium group; Thermotogales, Thermus/Deinococcus group, etc.

Specific bacterial species of interest include, but are not limited to Gram-negative bacteria such as Citrobacter sp.; Enterobacter sp.; Escherichia sp., e.g. E. coli; Klebsiella sp.; Morganella sp.; Proteus sp.; Providencia sp.; Salmonella sp., e.g. S. typhi, S. typhimurium, Serratia sp.; Shigella sp.; Pseudomonas sp., e.g. P. aeruginosa; Yersinia sp., e g. Y pestis, Y. pseudotuberculosis, Y enterocolitica, Franciscella sp.; Pasturella sp.; Vibrio sp., e.g. V. cholerae, V. parahemolyticus; Campylobacter sp., e.g. C. jejuni; Haemophilus sp., e.g. H. influenzae, H. ducreyi; Bordetella sp., e.g. B. pertussis, B bronchiseptica, B. parapertussis; Brucella sp., Neisseria sp., e.g. N. gonorrhoeae, N. meningitidis, etc. Other bacteria of interest include Legionella sp., e.g. L. pneumophila, Listeria sp., e.g. L. monocytogenes;, Mycoplasma sp., e.g. M hominis, M. pneumoniae; Mycobacterium sp., e.g. M. tuberculosis, M. leprae, Treponema sp., e.g. T pallidum; Borrelia sp., e.g. B. burgdorferi; Leptospirae sp.; Rickettsia sp., e.g. R. rickettsit, R. typhi, Chlamydia sp., e.g. C. trachomatis, C. pneumoniae, C. psittaci; Helicobacter sp., e.g. H. pylori, etc.

Eukaryotic organisms of interest include various fungal, plant, animal and protozoan groups. These may include, without limitation Acanthamoebidae; Acantharea, Ciliophora (ciliates); Dinophyceae (dinoflagellates), Euglyphina, Cryptophyta (cryptomonads); Diplomonadida (diplomonads); Entamoebidae, Euglenozoa; Fungi group, e.g. Choanoflagellida, Acanthoecidae, Codonosigidae, Ascomycota (ascomycetes), Basidiomycota (basidiomycetes), Chytridiomycota, Zygomycota, etc.; Mycetozoa, e.g. Dictyosteliida (dictyostelid cellular slime molds), Acytostelium, Dictyostelium, Polysphondylium, etc.; Parabasalidea (parabasalids), e.g. Trichomonadida (trichomonads), Dientamoeba, Trichomonas, Tritrichomonas, etc., Paramyxea, Plasmodiophorida; Polycystinea; Rhodophyta (red algae), e.g. Bacillariophyta (diatoms), Bacillariophyceae (Raphid, penn ate diatoms), Coscinodiscophyceae (centric diatoms), Fragilariophyceae (Araphid, pennate diatoms); Blastocystis; Dictyochophyceae; Eustigmatophyceae; Hyphochytriomycetes; Phaeophyceae (brown algae), mammalian tumor cells, liver cells, umbilical cord cells, fetal cells, etc.

Pathogenic fungal species of interest include, but are not limited to Magnaporthe grisea; Mycosphaerella graminicola; Ustilago maydis; Ophiostoma novo-ulmi; Botryotinia fuckeliana; Colletotrichum lagenarium; Rhizoctonia solani, Aspergillus fumigatus, Candida albicans, and the like.

Other fungal species can include Aspergillus niger; Aspergillus nidulans; Neurospora crassa and Saccharomyces cerevisiae.

The cells may be naturally occurring, or may be genetically modified, in order to determine the effect of a test compound in conjunction with a targeted gene or pathway. As used herein, the term “genetic modification” refers to an experimentally introduced change in the genetic composition of a cell (generally through the introduction of a vector comprising exogenous DNA, into a cell). The resulting, modified cell will have increased or decreased expression of the genetic sequence.

Introduction of an expression vector encoding a polypeptide is used to express the encoded product in cells lacking the sequence, or to over-express the product. These coding sequences may include full-length coding sequences, fragments derived therefrom, or chimeras that combine a naturally occurring sequence with functional or structural domains of other coding sequences. Alternatively, the introduced sequence may be an anti-sense sequence, dsRNA, encode a dominant negative mutation, or dominant or constitutively active mutations of native sequences; altered regulatory sequences, etc.

The introduced sequences may be a wild-type sequence, i.e. the sequence of a gene or part of a gene that is generally found in normal individuals of the host species. In some instances two or more polymorphic variants may be considered “wild type” genes, as is known in the art. Alternatively the sequence may be a naturally occurring or experimentally created mutation, or other alteration in the sequence.

In addition to sequences derived from the host cell species, other sequences of interest include, for example, genetic sequences of pathogens, for example coding regions of viral, bacterial and protozoan genes, particularly where the genes affect the function of human or other host cells. Sequences from other species may also be introduced, where there may or may not be a corresponding homologous sequence. Introduced sequences may also be transposons or other DNA integrated into a specific location within the genome by homologous recombination.

A variety of methods may be used to achieve a decrease in gene expression or activity, herein generically referred to as a “knock-outs”, including site-specific recombination, expression of anti-sense or dominant negative mutations, and the like. Knockouts have a partial or complete loss of function in one or both alleles (if present) of the endogenous gene in the case of gene targeting. Preferably the target gene expression is undetectable or insignificant in the cells being analyzed. This may be achieved by introduction of a disruption of the coding sequence, e.g. insertion of one or more stop codons, insertion of a DNA fragment, etc., deletion of coding sequence, substitution of stop codons for coding sequence, etc. In some cases the introduced sequences are ultimately deleted from the genome, leaving a net change to the native sequence.

Methods which are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

Assay conditions: For the purposes of the invention, cells are generally grown in a minimal medium supplemented with one or more single nutrients (or substrates for growth), where each combination of test compound, organism, media and nutrients provides for a single assay condition, and where a plurality of assay conditions are used in the subject screening methods. By plurality, it is meant at least about 10, more usually at least about 50, and preferably at least about 95 assay conditions, and may comprise multiple sets of 95 assay conditions. U.S. Pat. Nos. 6,046,021 and 5,627,045, the contents of which are incorporated herein, teach methods and compositions for growing, characterizing and testing of microorganisms in a multitest format.

In one embodiment, the growth in the well where the organism is capable of using the nutrient results in a color change from an indicator dye, e.g. via tetrazolium reduction. Color indicators may be chromogenic substrates, oxidation-reduction indicators, pH indicators, etc. In alternate preferred embodiments, the oxidation-reduction indicator is tetrazolium violet, while in other embodiments, the oxidation-reduction indicator is redox purple. Chromogenic pH indicators, include, but are not limited to such compounds as bromthymol blue, bromcresol purple, and neutral red. In a preferred embodiment, growth in the well is determined by measuring turbidity.

The medium may also comprise a gelling agent, including, but not limited to agar, pectin, carrageenan, alginate, alginic acid, silica, gellans and gum. In one embodiment, the pectin medium of Roth (U.S. Pat. Nos. 4,241,186, and 4,282,317; herein incorporated by reference) is used. In another embodiment, the gellan of Kang et al. (U.S. Pat. Nos. 4,326,052 and 4,326,053, herein incorporated by reference) is used. Phytagel™, a gellan gum, is the preferred gelling agent. In each embodiment, the cells to be tested are mixed in a suspension comprising a gelling agent, and then inoculated into a well, compartment, or other receptacle, which contains the biochemical(s) to be tested, along with a gel-initiating agent such as various cations. Upon contact of the gelling agent with the gel-initiating agent (e.g., cations), the suspension solidifies to form a viscous colloid or gel, with the cells evenly distributed throughout.

Compounds or genetic changes that result in auxotrophy cause the strain to fail to grow in all wells except the one containing the necessary nutrient. Alternatively, compounds or genetic changes may allow growth in the presence of some nutrients and not others. In some cases, the wells contain more than one nutrient, in order to allow analysis of genes or compounds that affect more than one biosynthetic pathways (e.g., isoleucine+valine (ilv), arginine+uracil (car), and purine+pyrimidine+histidine+tryptophan+nicotinamide (prs)).

In an alternative embodiment, a “drop out” medium or substrate is used. In this system, a complex defined supplement is used and one nutrient is missing in the substrate dispensed in each well (i.e., the medium lacks one nutrient of the substrate complex).

A wide range of compounds may be used as carbon sources. Sugars are commonly used in microbial testing panels, e.g. glucose, fructose, sucrose, maltose, ribose, erythrose, arabinose, threose, xylose, galactose, talose, gulose, mannose, altrose, allose, sorbose, lactose, etc.

In addition, amino acids may be included as carbon sources, e.g. D- and L- alanine, D- and L- serine, D- and L-threonine, D- and L-aspartate, L-asparagine, L-glutamine, L-glutamate, and L-proline and the like.

The present invention also provides methods and compositions to observe utilization of nitrogen, phosphorus, and sulfur sources, using an indicator system, e.g. tetrazolium reduction, to demonstrate substrate utilization. Nitrogen sources include, without limitation, D-alanine, L-alanine, L-arginine, D-asparagine, L-asparagine, D-aspartic acid, L-aspartic acid, L-cysteine, L-cystine, D-glutamic acid, L-glutamic acid, L-glutamine, glycine, L-histidine, L-homoserine, D,L-B-hydroxy-glutamic acid, L-isoleucine, L-leucine, L-phenylalanine, L-proline, D-serine, L-serine, L-tryptophan, L-tyrosine, glutathione (as well as any peptide containing the above amino acids), adenosine, deoxyadenosine, cytosine, cytidine, deoxycytidine, D-glucosamine, D-galactosamine, D-mannosamine, N-acetyl-D-glucosamine, N-acetyl-D-galactosamine, N-acetyl-D-mannosamine, methylamine, ethylamine, butylamine, isobutylamine, amylamine, ethanolamine, ethylenediamine, pentamethylenediamine, hexamethylenetriamine, phenylethylamine, histamine, piperidine, pyrrole, β-alanine, glycocoll, acetylglycocoll, phenylglycine-o-carbonic acid, hippuric acid, urocanic acid, α-aminovaleric acid, γ-aminovaleric acid, α-aminoisovaleric acid, γ-aminoisovaleric acid, α-aminocaproic acid, γ-aminocaprylic acid, acetamide, lactamide, glucuronamide, formamide, propionamide, methoxylamide, thio-acetamide, cyanate, urea, diethylurea, tetraethylurea, biuret, parabanic acid, alloxan, alloxantine, allantoin, uric acid, theobromine, guanine, and xanthine.

Phosphorous sources include, but are not limited to pyrophosphate, trimetaphosphate, 2′-mononucleotides, 3′-mononucleotides, 5′-mononucleotides, 2′, 3′-cyclic nucleotides, 3′, 5′-cyclic nucleotides, aryl-phosphates (e.g., p-nitrophenyl phosphate), phosphonates (e.g., aminoethyl phosphonate), sugar phosphates (e.g., glucose-1-phosphate), acid phosphates (e.g., 2-phospho-glyceric acid), aldehyde phosphates (e.g., glyceraldehyde-3 phosphate), α-glycerol phosphate, β-glycerol phosphate, inositol phosphates (e.g., phytic acid), phosphite, hypophosphite, and thiophosphate.

Sulfur sources include, without limitation, sulfur, thiosulfate, thiophosphate, metabisulfite, dithionite, tetrathionate, polysufide, cysteine, cystine, cysteic acid, cysteamine, cysteine sulphinic acid, cystathionine, lanthionine, ethionine, methionine, N-acetyl-methionine, N-acetyl-cysteine, glycyl-methionine, glycyl-cysteine, glutathione, L-djenkolic acid, L-2-thiohistidine, S-methyl-cysteine, S-ethyl-cysteine, methionine sulfoxide, methionine sulfone, taurine, thiourea, and thioglycolate.

In addition, various amino and carboxy peptidases can be tested for use in the present invention, by observing the utilization of but not limited to, dipeptides containing all natural L-amino acids on the amino terminal, and all natural L-amino acids on the carboxy terminal, as well as suitable non-protein occurring amino acids, such as pyroglutamate, ornithine, α-amino butyrate, D-amino acids, etc.

The present invention also provides methods and compositions for testing growth at extremes of pH, salinity and temperatures, and the compensatory effect of several compatible solutes. In addition, diauxic testing is performed with a limiting amount of a favored nutrient present in a well. In this embodiment, the cells need to adapt from a more favored to a less favored nutrient, and the lag and growth kinetics for numerous substrates can be measured quickly and efficiently in a microtiter plate format.

In one embodiment of the invention, each test compound will be provided in combination with many different assay conditions, e.g. one or more sets of 95 tests that are aimed toward each of the following groups of tests, which encompasses the majority of the catabolic functions of cells, as well as the majority of the biosynthetic functions of cells, and much of the macromolecular machinery of the cell including the ribosome, DNA and RNA polymerases, cellular respiration, transport and detoxification systems, cell wall, and inner and outer membranes: (1) carbon source oxidation tests (including peptide substrates), (2) carbon source fermentation tests, (3) amino and/or carboxy peptidase tests, (4) nitrogen source tests, (5) phosphorus source tests, (6) sulfur source tests; (7) auxotrophic tests for all essential metabolites such as amino acids, vitamins, polyamines, fatty acids, and/or nucleosides; (8) sensitivity tests for antibiotics and antimicrobials; (9) sensitivity tests for amino acid analogs, sugar analogs, nucleoside and base analogs, and/or mutagens; (10) sensitivity tests for dyes, detergents, heavy metals, oxidizing and/or reducing agents; and (11) other tests of general physiological interest such as growth at different pH concentrations, salt concentrations, temperatures, utilization of different osmotic balancers, and/or ability to traverse various diauxic “shift-downs.”

Microplate: The assay conditions of the present invention are conveniently provided in an indicator plate that is similar in structure to microtiter plates, which are commonly used in the art and commercially available from numerous scientific supply sources (e.g., Biolog, Fisher, etc.) In one embodiment, standard 96-well microtiter plates are used. In other embodiments, microtiter plates with more wells are used (e.g., 384 well and 1536 well microtiter plates). Furthermore, the microtiter plate format is suited for methods for kinetic analysis of substrate utilization by cells.

The present invention also contemplates a multitest indicator plate that is generally useful in the phenotypic characterization, as well as identification and antimicrobial sensitivity testing of microorganisms. This medium and method are particularly targeted toward some of the most economically important organisms, as well as species of clinical importance.

For example, a commercially available test panel (FF MicroPlate™, Biolog) may be used. This panel contains 95 carbon sources. To perform a test, identical cell suspensions of isogenic parental and mutant strains are prepared and pipetted into the 96 wells of a microplate. The cells are incubated for approximately 16-24 hours and if a substrate oxidation occurs in a given well, a violet/purple color is produced due to coupled reduction of a tetrazolium dye. Quantitation of the intensity of color is possible through use of a microplate reader or comparable instrument, or the plates can even be compared by eye. For observation of differences at a finer level, the MicroPlates™ can be read at frequent time intervals to determine the kinetics of color formation (i.e., carbon source oxidation rates) in each of the 96 wells. For a typical strain, perhaps 80 to 85 wells provide positive reactions and useful data. It should be noted that in some cases where fungi do not work well with indicator dyes, then turbidity measurements may be utilized.

An alternate embodiment of the invention utilizes a “microcard” (e.g. MicroCard™, Biolog) device for the multiparameter testing of chemical, biochemical, immunological, biomedical, or microbiological samples in liquid or liquid suspension form in a small, closed, easy-to-fill device, and is particular suitable for multiparameter testing and identification of microorganisms. For example, a microcard may be approximately 75 mm in width and 75 mm in length, and approximately 3 mm in depth. Approximately one-tenth the volume of cells are used to inoculate the compartments of the device, as compared to standard microplates. After the device has been filled, a non-venting, sealing tape can be applied to the device to cover the gas-venting, liquid barrier to reduce the evaporation of the liquid from the device. In some embodiments, the tape can permit the molecular diffusion of oxygen and/or carbon dioxide into or out of the device to maintain the desired chemical or biochemical environment within the device for successful performance of the test. Where the liquid receiving means comprises liquid entry ports, a similar closing tape can be applied to close the port or ports to prevent spilling and evaporation of the liquid therefrom.

With any of the testing formats, the visual result that is detected by eye or by instrument can be any optically perceptible change such as a change in turbidity, a change in color, a change in fluorescence, or the emission of light, such as by chemiluminescence, bioluminescence, or by Stokes shift. Color indicators may be, but are not limited to, redox indicators (e.g., tetrazolium, resazurin, and/or redox purple), pH indicators, or various dyes and the like. Various dyes are described in U.S. Pat. Nos. 4,129,483, 4,235,964 and 5,134,063.

Candidate compounds are screened for biological activity by adding the compound to one or more assay combinations. The change in cell growth or substrate utilization on various substrates in the presence of the compound is measured. Molecules of interest for analysis include any biologically active molecule with the capability of modulating directly or indirectly, the phenotype of a cell of interest. The information thus obtained is useful for drug discovery, identification of the target/mechanism of candidate drug action, facilitation of SAR (structure, action, and reaction) studies, rational approaches to efficacy improvement, and rapid categorization of compounds as to possible therapeutic applications and potential toxicity. The method of the invention can also provide a means to identify genes that play a role in, or that can influence, a selected biological process or pathway.

The compounds are typically added in solution, or readily soluble form, to the medium of cells in culture. Alternatively, cells are removed from a culture medium and suspended at a desired density in saline, water, gel, gelling agent, buffer, or solution. This suspension is then introduced into the compartments of the testing device which have been prefilled with basal medium, indicator, and substrate chemicals. The overall composition of the culture medium should not change significantly with the addition of the test compound or organism. Preferred compound formulations do not include additional components, such as preservatives, that have a significant effect on the overall formulation. Thus preferred formulations consist essentially of a biologically active compound and a physiologically acceptable carrier, e.g. water, ethanol, DMSO, etc. However, if a compound is liquid without an excipient the formulation may consist essentially of the compound itself.

A plurality of assays may be run in parallel with different compound concentrations to obtain a differential response to the various concentrations. As known in the art, determining the effective concentration of a compound typically uses a range of concentrations or dilution series in a range from about 1:2 to about 1:10, or other log scale, dilutions. The concentrations may be further refined with a second series of dilutions, if necessary. Typically, one of these concentrations serves as a negative control, i.e. at zero concentration or below the level of detection.

Following exposure to a testing substrate such as a carbon or nitrogen source, or an antimicrobial, the response of an organism may be detected, i.e. an output signal such as a change in chromogenic substrate or increase in growth measurements such as turbidity. This detection may be visual (i.e., by eye) or accomplished with the assistance of machine(s). For example, the response of organisms to carbon sources may be detected as turbidity in the suspension due to growth resulting from the utilization of the testing substrate by the organisms. Likewise, growth can be used as an indicator that an organism is not inhibited by certain antimicrobials. In one embodiment, color is used to indicate the presence or absence of organism growth/metabolism. An instrument system may be used that allows the reading of testing panels inoculated with cells, and analyzes the data obtained from the testing panels. This allows the rapid analysis of multiple phenotypic characteristics for many assay conditions in a short time.

The evaluation of each test plate, or sets of test plates, provides a series of values corresponding to the growth of the cells in each of the wells, where the wells provide the various environmental conditions. These values correlate with the effect of the test compound on the cell. The set of these values, (or data points), for a test compound or test organism can be summarized as a dataset, which may be referred to as a reference pattern, or a test pattern. Test patterns differ from reference patterns only for purposes of data handling, where the term “test pattern” is used to refer to a compound that is being analyzed, for example in the classification of an known test compound. The term “reference pattern” is generally used to refer to the information against which the test pattern is compared. It will be understood by one of skill in the art that the information content does not differ, and the terms merely provide a convenient means of discussing data analysis. The reference patterns find use in comparative studies, in structure function analysis, and the like.

The amount of information in a reference pattern can be expanded by performing additional tests. For any one compound, analyses can be performed by testing the compound against other cells or organisms; by comparing the compound to different controls or under different environmental conditions, e.g. salt concentration, pH, etc.; by comparing analogous compounds, and the like.

The reference (or test) pattern is comprised of an array of values obtained from the growth of the cells, oxidation, reduction or fermentation of nutrients, etc., for example each well may provide one or more optical density values corresponding to the indicator dyes present in the growth medium. These dataset values may be cross-referenced by the different parameters, e.g. the test compound that was added to the cells, the responding cell types, the negative controls, etc. Classification and identification of compounds can use this reference pattern for later comparison of test patterns.

A feature of the invention is the generation of a database of reference patterns for a variety of compounds. Such a database will typically comprise reference pattern information as described above, for a number of compounds, which may be related compounds. The compounds of interest in a database may be selected and arranged according to various criteria: the types of molecules that are tested, e.g. classes of antibiotics, antifungals, etc.; by the source of compounds, e.g. environmental toxins, biologically active extracts from a particular animal or cell, etc.

The reference patterns and databases thereof may be provided in a variety of media to facilitate their use. “Media” refers to a manufacture that contains the reference pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. One format for an output means ranks new test patterns possessing varying degrees of similarity to a reference pattern. Such presentation provides a skilled artisan with a ranking of similarities and identifies the degree of similarity contained in the test pattern.

The comparison of a test pattern obtained from a test compound and a reference pattern is accomplished by any suitable deduction protocol, AI system, statistical comparison, etc. Methods of searching databases are known in the art. See, for example, U.S. Pat. No. 5,060,143, which discloses a highly efficient string search algorithm and circuit, utilizing candidate data parallel, target data serial comparisons with an early mismatch detection mechanism. For other examples, see U.S. Pat. No. 5,720,009 and U.S. Pat. No. 5,752,019.

The data that is obtained from a test compound can be compared to a reference pattern(s) determine whether the signals can be superimposed, i.e. whether there is a match between any two patterns; or whether they vary. Since a reference pattern and test pattern may be comprised of multiple values, the matching may be performed with several different datasets.

Where an unknown compound is being tested against a single reference compound, the comparison will typically involve determination of whether there is a statistically significant match for the cell growth under different environmental conditions, e.g. in the presence of different carbon sources, etc. If the dataset from a test compound is being compared with multiple reference patterns, multiple comparisons for a single compound are provided, and then the determination will be for a statistically significant match for each of the comparisons. When an unknown compound is compared against a database, a more sophisticated search may be required to first determine the reference pattern that is the closest match to the modulation test pattern.

Methods of classifying compounds according to their action may utilize comparisons across different parameters than those that are used for identification of unknown compounds. The parameter of interest may not be whether a test pattern matches a reference pattern, but whether the compound shows a difference in, for example, growth in a specific carbon source, etc.

In one aspect of the invention, the evaluation of a test pattern finds use in determining the microbial pathway being affected by a particular compound. By analysis of the growth of the microbe it can be determined which biosynthetic pathways are affected by the test compound. Further information can be obtained by comparison with reference patterns, e.g. the growth patterns of the same organism in the presence of compounds with a known mode of action.

The evaluation and comparison of test patterns find use in the classification of biologically active compounds, e.g. for the evaluation of drug candidates, herbicides, antifungals, etc. For these purposes it is of interest to determine the effect of a test compound against different cells; and/or the range of effects in a family of compounds. For example, a series of analogs may be made to a known antibiotic, and the effect of this series of analogs determined by the subject methods.

In another embodiment, the evaluation and comparison of test patterns is used to identify unknown compounds. Such methods find particular use in conjunction with, for example, environmental testing, or testing of novel natural products. Environmental samples may comprise potential toxins or other biological hazards, where it is desirable to identify as quickly as possible the contaminants. Natural product samples may comprise pharmaceutically active drugs, such as anti-microbials or anti-fungal drugs, and by-products derived therefrom, and the like.

Where the sample to be tested may comprise a complex mixture of biologically active compounds, the identification process may utilize a subtraction step. However, in addition to obtaining the comparison of the cell growth against, e.g. a negative control, the values may also be compared to a “normal” sample, e.g. uncontaminated water; or other biological samples similar to that being tested, in order to isolate the effect of the potential test compound.

Databases of reference patterns are developed using the above-described techniques, where such databases find use as a resource for the classification and identification of compounds. In addition, the mode of action of a compound can be determined by comparing compound reference data and data derived from genetically modified organisms.

It is to be understood that this invention is not limited to the particular methodology, protocols, microbial species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

### Experimental

MIC Determination and Test Parameters

The initial step for determining the effect of fungicides on fungi is the determination of the minimal inhibitory concentration (MIC) of the fungicides. MICs were determined for both M. grisea and S. cerevisiae. These analyses were performed in a microtiter plate format modified from National Committee for Clinical Laboratory Standards, Document M27. Fungal conidia or cells are suspended in rich liquid media at a specific concentration, and dispensed into the wells of a microtiter plate. A separate microtiter plate containing fungicides in a two fold dilution series was used as a source plate for adding fungicides to the inoculated plate. negative controls and solvent controls were included in each microtiter plate. The plates were incubated at the appropriate temperature and time dependent on each fungal species. The MIC is determined by both visual inspection of the wells and by spectrophotometer analysis (OD₆₀₀) of the plates. The MIC is defined as the lowest concentration at which complete inhibition of fungal growth was seen. For inhibitors of melanin biosynthesis (tricyclazole and pyroquilon) no growth inhibition is observed by spectrophotometric analysis. For these fungicides, the MIC is defined as the lowest concentration at which melanin production is inhibited as determined by visual analysis of the microtiter plate wells.

Fungicide treatments for nutritional profiling analysis is performed using two fungicide concentrations, inhibitory (high) and sub-inhibitory (low). The high concentration, defined as 2-4×MIC, completely inhibits fungal growth. The low concentration was defined as 2-4 fold lower than the highest concentration at which no inhibition of growth was determined in the spectrophotometric analysis. It is expected that the addition of a sub-inhibitory concentration of fungicide will still reveal changes relating to the mode of action of the fungicide for compounds where the metabolism of specific compounds are affected by the chemical treatment.

The YT microplate test panel (Biolog, Inc.) is designed as a tool for identifying a broad range of yeasts by their carbon utilization patterns. Initial tests were performed with S. cerevisiae in these plates with and without the addition of various fungicides. Very simply, yeast cells are prepared as a water suspension as per the YT microplate instructions. Prior to loading the plates with cells, a fungicide or solvent control is added to the water suspension. The cells are immediately loaded onto a plate. The plates were incubated at 26° C. as per instructions, and OD₄₉₀ and OD₇₅₀ readings were obtained at various time points during incubation. The results of fungicide treated samples and controls are compared. Differences in well growth that is altered relative to the control is recorded and is presumed to relate to the mode-of-action of the chemical.

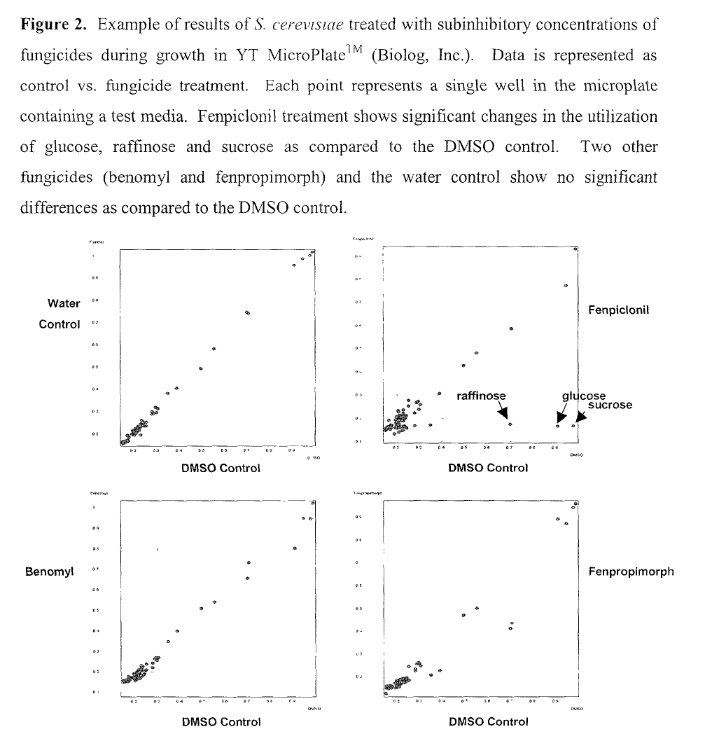

A test in the YT microplate was set up using the sub-inhibitory concentration of three fungicides for which a putative MOA is known. Two fungicides, benomyl and fenpropimorph are predicted to have no effect on carbon utilization, but the third, fenpiclonil, is predicted to affect glucose uptake and utilization. The experiments were performed as described. The results from 48 hours incubation are shown in FIG. 2. As predicted, benomyl and fenpropimorph did not result in any significant differences from the controls. Also as predicted from MOA reports, fenpiclonil showed a decreased utilization of glucose, raffinose and sucrose (raffinose and sucrose contain glucose as a subunit). These data show that by addition of fungicides to cells prior to loading onto biolog Microplates, alterations in nutrient utilization in the presence of chemicals can be found and may lead to a better understanding of the chemicals MOA.

This data provides support for the utilization of a microtiter plate nutrient utilization induced by the presence of chemical compounds. The compounds for testing include fungicides, herbicides, insecticides, human anti-fungals, antibiotics, disinfectants, etc. The tests described above utilize a carbon utilization plate, but plates for testing a nearly unlimited number of nutrients could be used, including nitrogen, phosphate and sulphate sources, for which Biolog Inc. has already made plates for bacterial and fungal use. In addition, plates to test the ability of a fungus to bypass the effect of fungicide by providing a nutrient that is limited by the presence of a chemical compound, i.e. one that inhibits a specific biosynthetic pathway, can also be tested in this format. Biolog also produces an auxotrophy plate that can be used for this purpose.

In addition to fungi, any organism amenable to growth in microtiter plates can be analyzed in the presence of chemicals in a similar manner.

What is claimed is:

1. A method for characterization of a test compound, the method comprising:
contacting a cell with said test compound in the presence of a plurality of assay conditions, each comprising culture medium and a substrate for growth of said cell;
recording the growth or substrate utilization of said cell in the presence of said test compound; and
deriving a test pattern from the output signal resulting from contact between said cell and said test compound, wherein said test pattern indicates the phenotype of said cell in the presence of said test compound.

2. The method of claim 1, wherein said test pattern derived from said test compound is compared to a reference pattern obtained from a second test compound, wherein a variation between said test pattern and said reference pattern indicates a difference between cell substrate utilization or growth.

3. The method of claim 1, wherein said substrate utilization comprises one or more of carbon source oxidation tests; carbon source fermentation tests; amino and/or carboxy peptidase tests; nitrogen source tests; phosphorus source tests; sulfur source tests; auxotrophic tests for all essential metabolites; sensitivity tests for antibiotics and antimicrobials; sensitivity tests for amino acid analogs, sugar analogs, nucleoside and base analogs, and/or mutagens; sensitivity tests for dyes, detergents, heavy metals, oxidizing and/or reducing agents; growth at different pH concentrations; growth at different temperatures; growth at different salt concentrations; utilization of different osmotic balancers; and ability to traverse diauxic shift-downs.

4. The method of claim 3, wherein said contacting is performed in a microplate format.

5. The method of claim 3, wherein said contacting is performed in a microcard format.

6. The method of claim 1 wherein said test compound is an unknown compound.

7. The method of claim 1 wherein said test compound is a known compound.

8. The method of claim 1 wherein said test compound is present in an environmental sample.

9. The method of claim 1 wherein said test compound is a drug candidate.

10. The method of claim 2, wherein said comparison is performed against a database of reference patterns determined for a plurality of compounds.

11. The method of claim 10, wherein said comparison provides for classification of said test compound.

12. The method of claim 11, wherein said the analysis of turbidity and/or chromagenic substrates of a subset of said plurality of compounds provides for determination of the mode of action of said test compound.

13. The method of claim 1, wherein said cell is selected from the group consisting of a fungal cell, a bacterial cell, a plant cell, an algal cell, a protozoan cell and an animal cell.

14. The method of claim 1, wherein said cell is a fungal cell.

15. The method according to claim 1, wherein said cell is a plant cell.

16. A method for characterization of a polynucleotide sequence, the method comprising:
genetically modifying a cell with said polynucleotide sequence;
contacting at least one of said genetically modified cell with a plurality of assay conditions, each comprising culture medium and a substrate for growth of said cell;
recording the growth or substrate utilization of said cell; and
deriving a test pattern from the output signal resulting from contact between said cell and said assay conditions, wherein said test pattern indicates the phenotype of said cell resulting from genetic modification with said polynucleotide sequence.

17. The method of claim 16, wherein said test pattern derived from said test compound is compared to a reference pattern obtained from one or more of: a wild-type cell, a cell genetically modified with a second polynucleotide sequence, or a cell grown in the presence of a test compound, wherein a variation between said test pattern and said reference pattern indicates a difference between cell substrate utilization or growth.

18. The method of claim 16, wherein said substrate utilization comprises one or more of carbon source oxidation tests; carbon source fermentation tests; amino and/or carboxy peptidase tests; nitrogen source tests; phosphorus source tests; sulfur source tests; auxotrophic tests for all essential metabolites; sensitivity tests for antibiotics and antimicrobials; sensitivity tests for amino acid analogs, sugar analogs, nucleoside and base analogs, and/or mutagens; sensitivity tests for dyes, detergents, heavy metals, oxidizing and/or reducing agents; growth at different pH concentrations; growth at different temperatures; growth at different salt concentrations; utilization of different osmotic balancers; and ability to traverse diauxic shift-downs.

19. The method of claim 18, wherein said contacting is performed in a microplate format.

20. The method of claim 18, wherein said contacting is performed in a microcard format.

21. The method of claim 17, wherein said comparison is performed against a database of reference patterns determined for a plurality of compounds.

22. The method of claim 10, wherein said comparison provides for classification of said polynucleotide sequence.

23. The method of claim 16, wherein said cell is selected from the group consisting of a fungal cell, a bacterial cell, a plant cell, an algal cell, a protozoan cell and an animal cell.

24. The method of claim 16, wherein said cell is a fungal cell.

25. The method of claim 16, wherein said cell is a plant cell.

26. A database of reference patterns, said database comprising a plurality of reference patterns obtained for one or more test compounds by the method of:
contacting at least one cell with said test compound in the presence of a plurality of assay conditions, each comprising culture medium and a substrate for growth of said cell;
recording the growth or substrate utilization of said cell in the presence of said test compound; and
deriving a test pattern from the output signal resulting from contact between said cell and said test compound, wherein said test pattern indicates the phenotype of said cell in the presence of said test compound.

27. A database of reference patterns, said database comprising a plurality of reference patterns obtained for one or more polynucleotide sequences by the method of:
genetically modifying a cell with said polynucleotide sequence;
contacting at least one of said genetically modified cell with a plurality of assay conditions, each comprising culture medium and a substrate for growth of said cell;
recording the growth or substrate utilization of said cell; and
deriving a test pattern from the output signal resulting from contact between said cell and said assay conditions, wherein said test pattern indicates the phenotype of said cell resulting from genetic modification with said polynucleotide sequence.
